(19) United States
(12) Patent Application Publication
(10) Pub. No.: US 2020/0188558 A1
(43) Pub. Date: Jun. 18, 2020

(54) HYDROGEL BASED ENCAPSULATION DEVICE

(71) Applicant: Mayo Foundation for Medical Education and Research, Rochester, MN (US)

(72) Inventors: Yogish C. Kudva, Rochester, MN (US); Amit L. Garle, Rochester, MN (US); Dennis A. Wigle, Rochester, MN (US); Yasuhiro Ikeda, Rochester, MN (US); Michael J. Yaszemski, Rochester, MN (US)

(73) Assignee: Mayo Foundation for Medical Education and Research, Rochester, MN (US)

(21) Appl. No.: 16/620,639

(22) PCT Filed: Jun. 12, 2018
(86) PCT No.: PCT/US2018/037150

§ 371 Date: Dec. 9, 2019

Related U.S. Application Data
(60) Provisional application No. 62/518,341, filed on Jun. 12, 2017.

(51) Int. Cl. A61L 27/52; A61L 27/54; A61L 27/18; C08L 75/04; A61L 27/38

(52) U.S. Cl. CPC A61L 27/52; A61L 27/54; A61L 27/3834; C08L 75/04; A61L 27/18

(57) ABSTRACT

Encapsulation devices comprising hydrogels are provided. Methods of making the encapsulation devices and methods of using the devices to provide implant cells, treat a disease, and prevent immunologic attack on implanted material are also provided.

### CROSS-REFERENCE TO RELATED APPLICATIONS

This application claims priority to U.S. Application Ser. No. 62/518,341, filed on Jun. 12, 2017. The disclosure of the prior application is considered part of the disclosure of this application, and is incorporated in its entirety into this application.

### TECHNICAL FIELD

This document relates to encapsulating biological material such as cells. This document also relates to implantable devices and methods for encapsulating biological material in hydrogels.

### BACKGROUND

Implantation of biological material such as cells shows promise in treating a variety of diseases. Encapsulation devices for implantation are often limited by immune attack, transferability of therapeutic products and nutrients through the encapsulation material, mechanical properties, and production conditions.

### SUMMARY

Provided herein are methods and devices for encapsulating and implanting biological material such as cells into a subject in need thereof.

Provided herein are hydrogel devices for encapsulating and implanting biological material such as cells into a subject in need thereof. In some embodiments, the devices are macro-sized devices. In some embodiments, the devices include a hydrogel film defining a cavity and having one or more openings therein. Also provided herein are processes for making the hydrogel devices, methods for implanting cells into a patient in need thereof using a device described herein, methods for treating a disease, such as type 1 diabetes, by implanting a device described herein into a patient in need thereof, and methods of preventing immunologic attack on implanted biological material using a device described herein.

The devices and methods described herein can provide several advantages. For example, the macro-sized devices described herein can provide larger loading volumes for cells while remaining small enough for implantation. High loading capacity can allow implantation of cell levels sufficient to exhibit therapeutic levels of cell products when implanted in vivo for disease treatment. Gelling or curing the hydrogels prior to loading with cells can avoid limitations in cell survival due to curing conditions and can provide an immune barrier for the cells. Gelling or curing the hydrogels prior to loading with cells can also allow a broad range of curing chemistries to be used, including, but not limited to toxic catalysts, higher temperatures, and higher chemical concentrations, that would otherwise be unavailable when cells are present during curing. The encapsulation devices can protect encapsulated cells from an implant host's immune system, while maintaining permeability sufficient to enable transport of cellular products across the encapsulating hydrogel membranes. In some cases, the devices described herein can provide high surface hydrophilicity and low protein binding. In some cases, the devices described herein can exhibit reduced inflammation, phagocytosis, protein adsorption, and thrombus formation when implanted in vivo. The composition of the hydrogels used herein can provide strong yet flexible mechanical properties. The hydrogel compositions can be tunable to adjust permeability to nutrients and therapeutic cell products, and can enable transport and vascularization at the perimeter of the device for oxygen and nutrient supply.

In one aspect, a device is provided, comprising a hydrogel construct including a hydrogel film having integral zwitterionic functional groups; a cavity defined by the construct; and one or more openings in the construct. The hydrogel film can comprise a single or multiple network hydrogel. The hydrogel film, in some embodiments, does not contain biological cells. In some embodiments, the hydrogel film can consist essentially of hydrogel. The hydrogel construct can be operable to allow permeation of solutes having a size of 100 kD or less through the hydrogel film while minimizing permeation of solutes having a size greater than 100 kD. The device can further comprise one or more sealing members operable to close at least one of the openings. The sealing member can be physical seals or chemical seals. The device can further comprise biological material positioned within the cavity. The biological material can, in some embodiments, comprise biological cells. In some embodiments, the biological material can comprise cells of at least one cell type selected from induced pluripotent stem cells (IPSC), insulin-secreting beta islet cells, glucagon-secreting alpha islet cells, and combinations thereof. The device can, in some embodiments, be expandable. In some embodiments, the device can be refillable.

In another aspect, a process for forming an encapsulation device is provided, comprising forming a hydrogel precursor film into a pouch having a cavity defined by the hydrogel precursor film and one or more openings in the hydrogel precursor film in fluid communication with the cavity. The process can further comprise soaking the hydrogel precursor film in a solution comprising a monomer, a crosslinker, and an initiator to form a soaked hydrogel precursor film; and polymerizing the soaked hydrogel precursor film to form a hydrogel film. The encapsulation device can be biocompatible. The cavity can operable to receive biological material, such as biological cells. In some embodiments, the biological material can comprise cells of at least one cell type selected from induced pluripotent stem cells (IPSC), insulin-secreting beta islet cells, glucagon-secreting alpha islet cells, and combinations thereof. The process can further comprise physically or chemically sealing the one or more openings. Forming a hydrogel film can comprise solution casting the hydrogel film. Forming a hydrogel film into a pouch can comprise solution casting the hydrogel on a mold by immersing at least a portion of the mold in a hydrogel solution to, thereby forming a continuous hydrogel film having one or more openings. Forming a hydrogel film into a pouch can comprise sandwiching a separator between a first surface of a first hydrogel film and a first surface of a second hydrogel film such that a first portion of the first surface of the first hydrogel film does not contact a first portion of the first surface of the second hydrogel film and a second portion of the first surface of the first hydrogel film contacts a second portion of the first surface of the second hydrogel film. The process can further comprise inserting biological cells into the cavity.

In another aspect, a method is provided for implanting cells into a patient comprising implanting into a patient in need thereof a device as disclosed herein.

In another aspect, a method is provided for treating a disease comprising implanting into a patient in need thereof a device as disclosed herein. The disease can, in some embodiments, be type 1 diabetes. In some embodiments, the disease can be type 2 diabetes.

In another aspect, a method of preventing immunologic attack on implanted biological material is provided, comprising encapsulating the biological material into a device as provided herein, and implanting the device into a subject in need thereof.

Unless otherwise defined, all technical and scientific terms used herein have the same meaning as commonly understood by one of ordinary skill in the art to which this invention pertains. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

For the terms “for example” and “such as,” and grammatical equivalences thereof, the phrase “and without limitation” is understood to follow unless explicitly stated otherwise. As used herein, the term “about” is meant to account for variations due to experimental error. As used herein, the singular forms “a,” “an,” and “the” are used interchangeably and include plural referents unless the context clearly dictates otherwise.

Also herein, the recitations of numerical ranges by endpoints include all numbers subsumed within that range (e.g., 1 to 5 includes 1, 1.5, 2, 2.75, 3, 3.80, 4, 5, etc.).

Other features and advantages of the invention will be apparent from the following detailed description and figures, and from the claims.

### DESCRIPTION OF DRAWINGS

FIG. 1A is a top view of an embodiment provided herein.

FIG. 1B is a vertical cross-sectional view of the embodiment of FIG. 1A taken at line X_{A}-X_{A}.

FIG. 1C is a side view of the embodiment of FIG. 1A taken at line A₁-A₁.

FIG. 1D is a top view of an embodiment provided herein.

FIG. 1E is a vertical cross-sectional view of the embodiment of FIG. 1D taken at line X_{D}-X_{D}.

FIG. 1F is a side view of the embodiment of FIG. 1B taken at line D₁-D₁.

FIG. 2A is a top view of an embodiment provided herein.

FIG. 2B is a side view of the embodiment of FIG. 2A taken at line A₂-A₂.

FIG. 2C is a top view of an embodiment provided herein.

FIG. 2D is a side view of the embodiment of FIG. 2C taken at line C₂-C₂.

FIG. 3A is a perspective view of an embodiment provided herein and a process of forming an embodiment provided herein.

FIG. 3B is a vertical cross-sectional view of the embodiment of FIG. 3A taken at line X₃-X₃.

FIG. 3C is a side view of the embodiment of FIG. 2A taken at line A₃-A₃.

FIG. 4A is a flow chart of a method according to an embodiment provided herein.

FIG. 4B is a flow chart of a method according to an embodiment provided herein.

FIG. 5 is a graph illustrating membrane thickness of selected hydrogels fabricated according to Example 1.

FIG. 6 is a graph illustrating membrane thickness of selected hydrogels fabricated according to Example 1.

FIG. 7 is a graph illustrating water content of selected hydrogels fabricated according to Example 1.

FIG. 8A is a graph illustrating diffusion of FITC dextran through hydrogels according to Example 2. Percent diffusion is shown on the y-axis.

FIG. 8B is a graph illustrating diffusion of rhodamine b dextran through hydrogels according to Example 2. Percent diffusion is shown on the y-axis.

FIG. 9A is a graph illustrating diffusion of FITC dextran through hydrogels according to Example 2. Percent diffusion is shown on the y-axis.

FIG. 9B is a graph illustrating diffusion of rhodamine b dextran through hydrogels according to Example 2. Percent diffusion is shown on the y-axis.

FIG. 10 is a schematic of membrane fabrication by dip coating, showing the progression through steps (i), (ii), and (iii). (i) Dipping of the rod. (ii) Withdrawing of the rod. Wet layer formation. (iii) Evaporation of solvent and formation of the tube.

FIG. 11A is a photograph of a hydrogel fabricated by dip coating according to Example 1.

FIG. 11B is a graph illustrating membrane thickness of selected hydrogels fabricated according to Example 1, including HG90.

FIG. 12A is a graph illustrating membrane thickness of selected hydrogels fabricated according to Example 1, including HG90.

FIG. 12B is a graph illustrating membrane thickness of selected hydrogels fabricated according to Example 1, including HG70.

FIG. 13 is a graph illustrating water content of selected hydrogels fabricated according to Example 1, including BG90/80=1:1

FIG. 14A is a cryo-SEM image of a HG90 hydrogel fabricated according to Example 1.

FIG. 14B is a cryo-SEM image of a BG90/80=1:1 hydrogel fabricated according to Example 1.

FIG. 14C is a cryo-SEM image of a HG80 hydrogel fabricated according to Example 1.

FIG. 15A is a graph illustrating cell viability with catalyst treatment of varying durations to Example 3.

FIG. 15B-D are fluorescence microscopy images of cell viability with catalyst treatment of varying durations according to Example 3.

FIG. 15E-G are bright-field microscopy images of cell viability with catalyst treatment of varying durations according to Example 3.

FIG. 16 is a graph of the water content of hydrogels including MPC fabricated according to Example 4.

FIG. 17 is a graph of the water content of hydrogels including MPC, CBMA, or SBMA fabricated according to Example 4.

FIG. 18A is a graph illustrating the tensile modulus of sterilized and unsterilized hydrogels fabricated according to Example 5.

FIG. 18B is a graph illustrating the tensile modulus of sterilized hydrogels fabricated according to Example 5.

FIG. 19A is a graph illustrating the protein adsorption of hydrogels fabricated according to Example 6.

FIG. 19B is a graph illustrating the protein adsorption of hydrogels using different concentrations of protein according to Example 6.

Like reference symbols in the various drawings indicate like elements.

### DETAILED DESCRIPTION

This document provides methods and devices for encapsulating biological cells. In some embodiments, hydrogel devices for encapsulating biological cells are provided. In some embodiments, the devices can be macrodevices. In some embodiments, the devices can be fabricated from any biocompatible hydrogel. In some embodiments, the devices can be fabricated from polyurethane hydrogels. In some embodiments, the devices can be fabricated from a hydrogel having integral zwitterionic functional groups, such as phosphocholine groups. In some embodiments, the devices can be fabricated from 2-methacryloyloxyethyl phosphorylcholine (MPC). In some embodiments, the devices can be fabricated from sulfobetaine methacrylate (SBMA). In some embodiments, the devices can be fabricated from carboxybetaine methacrylate (CBMA). In some embodiments, the devices can be fabricated from two or more hydrogels. In some embodiments, the devices include a multinetwork hydrogel film fabricated from two or more hydrogels. In some embodiments, the first network in a multinetwork hydrogel film can be a polyurethane (PU) network. In some embodiments, the second network in a hydrogel film can be a MPC, SMBA, or CBMA network. The devices can include a hydrogel construct defining a cavity and one or more openings in the hydrogel construct.

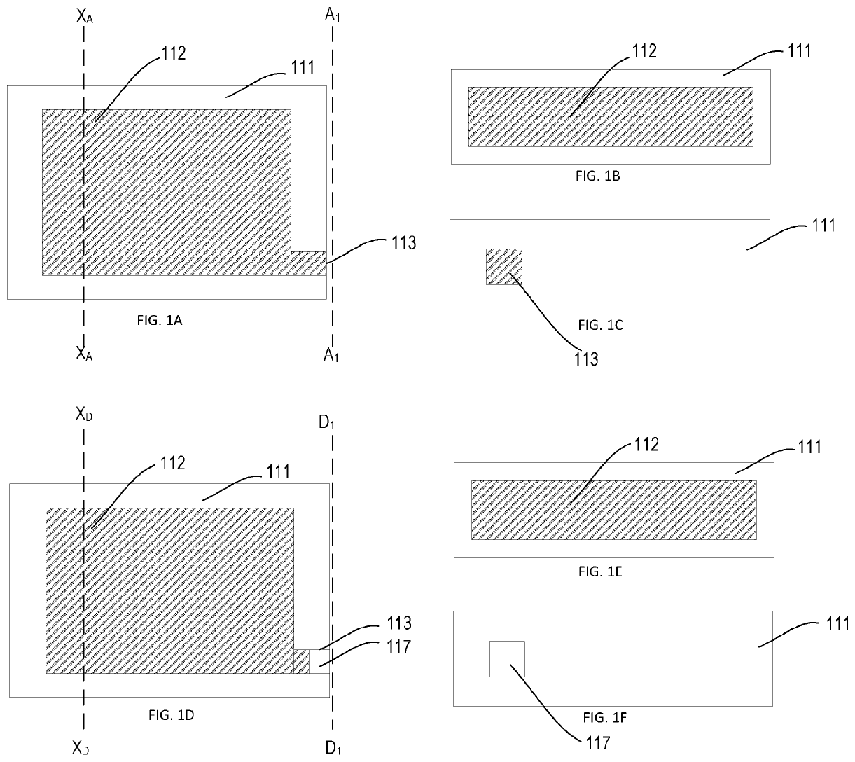

As illustrated in FIG. 1A-1F, in one embodiment, a hydrogel device 110 includes a hydrogel construct 111 comprising a hydrogel film. The hydrogel construct 111 defines a cavity 112 within the hydrogel construct. An opening 113 in the hydrogel construct 111 provides a port for filling cavity 112 with material such as biological cells. As shown in FIG. 1D-1F, in some embodiments, opening 113 can be sealed with a physical or chemical seal 117. Seal 117 can operate to close opening 113.

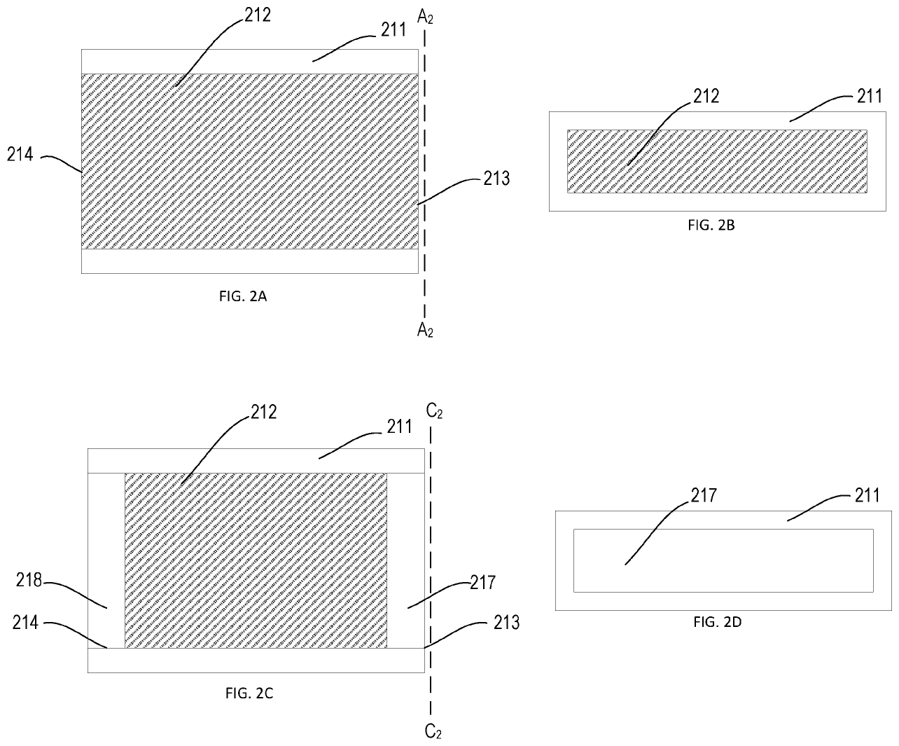

In another embodiment, as illustrated in FIG. 2A-2D, a hydrogel device 210 includes a hydrogel construct 211, a cavity 212 defined by the hydrogel construct 211, and two openings 213, 214 in the hydrogel construct. In some embodiments, one or both openings 213, 214 can be sealed with a physical or chemical seal 217, 218, as shown in FIG. 2C-2D. In some embodiments, a first opening 213 can be sealed with seal 217 prior to loading cells or other material into cavity 212. In some embodiments, following cell or material loading of cavity 212, a second opening 214 can be sealed with seal 218. In some embodiments, such as where polymerization or chemical bonding is used, seals 217 and 218 are indistinguishable from construct 211. In some embodiments, such as where physical seals are used, seals 217 and 218 are distinguishable from construct 211.

The document also provides a process for forming an encapsulation device. In some embodiments, the process includes forming a hydrogel precursor film. In some embodiments, the hydrogel precursor film is formed by solution casting. The hydrogel precursor film can be formed into a pouch to provide a cavity defined by the hydrogel precursor film. The pouch can include one or more openings in the film in fluid communication with the cavity. In some embodiments, forming the hydrogel precursor film and forming the hydrogel precursor film into a pouch can be completed in a single step, such as by solution casting the hydrogel precursor film onto a mold that provides a pouch shaped hydrogel precursor film. In some embodiments, hydrogel precursor films can be formed first, then the hydrogel precursor films can be shaped into a pouch shape or one or more hydrogel precursor films can be combined to form a pouch shape defining a cavity.

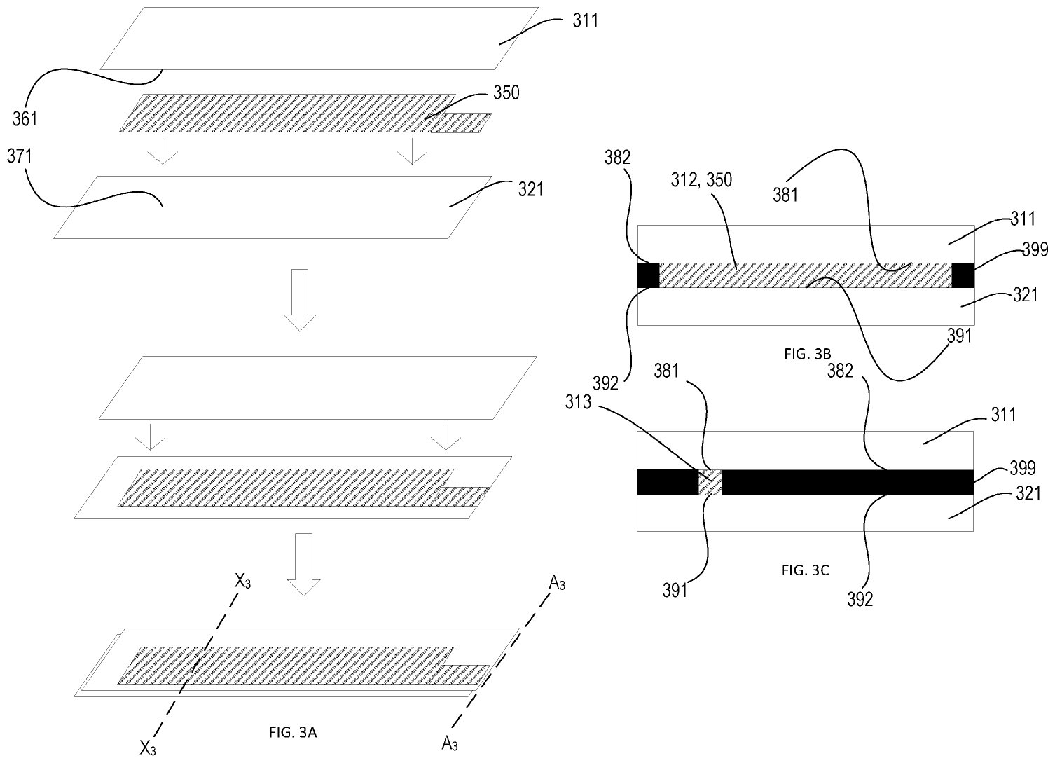

For example, in one embodiment shown in FIG. 3A-3C, a hydrogel precursor film pouch can be created by forming a first hydrogel precursor film 311 and a second hydrogel precursor film 321. As shown in FIG. 3, a separator 350 can be sandwiched between a first surface 361 of the first hydrogel film 311 and a first surface 371 of a second hydrogel film 321 such that a first portion 381 of the first surface 361 of the first hydrogel film 311 does not contact a first portion 391 of the first surface 371 of the second hydrogel film 321 and a second portion 382 of the first surface 361 of the first hydrogel film 311 contacts a second portion 392 of the first surface 371 of the second hydrogel film 321. The separator can aid in the formation of cavity 312 and opening 313. The structure can then be polymerized to chemically bond the second portion 382 of the first surface 361 of the first hydrogel film 311 to the second portion 392 of the first surface 371 of the second hydrogel film 321 through crosslinking at a contact portion 399. In some embodiments, contact portion 399 is a distinct portion of the construct comprising a polymerizable substrate. In some embodiments, contact portion 399 is indistinguishable from the rest of the construct, including films 311 and 321, after polymerization. After polymerization, the separator 350 can be removed. The polymerized structure results in a pouch comprised of a hydrogel construct defining a cavity 312 that can optionally include the separator 350.

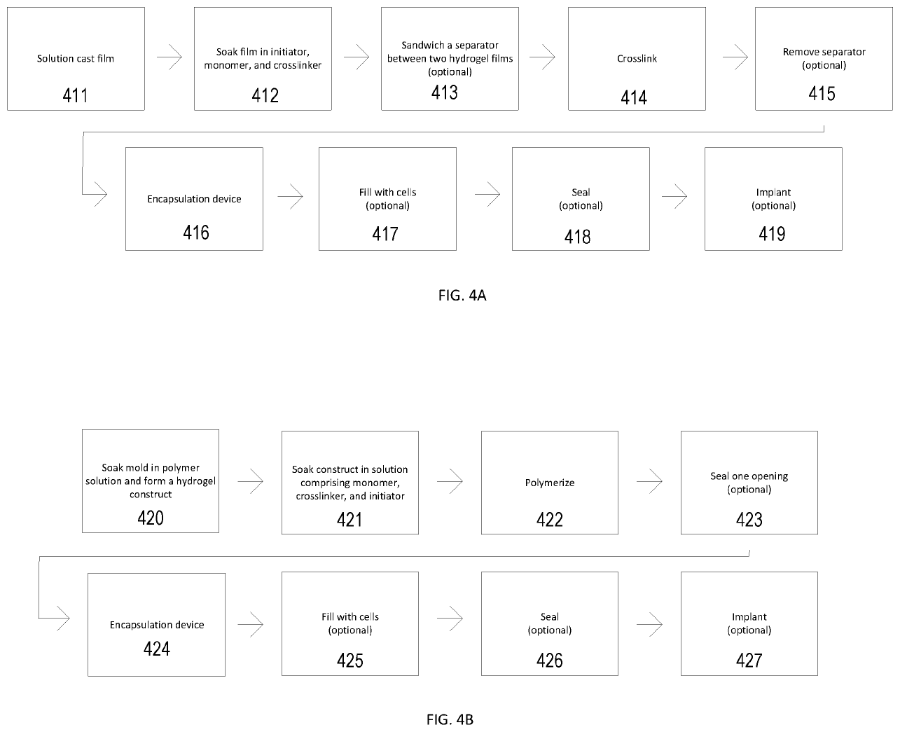

FIG. 4A further outlines a process 410 of forming an encapsulation device using two hydrogel precursor films. In step 411 the two or more precursor films can be solution cast. Then the films can be soaked in a solution of monomer, initiator, and crosslinker in step 412. A separator is optionally sandwiched between the two films in step 413 and the structure is crosslinked via, e.g., thermal or UV polymerization in step 414. In some embodiments, the order of steps 412 and 413 can be reversed. If present, the separator can optionally be removed (step 415), or retained. The resultant structure is an encapsulation device 416 having a cavity that can optionally be filled with cells or other material in step 417 through an opening or port in the hydrogel construct. Upon filling with cell or other material, the device can be sealed in step 418 to produce a filled, sealed encapsulation device or implant. The device can then optionally be implanted in step 419 into a subject by known methods of implantation.

In another embodiment, shown in FIG. 4B, a process for forming a device can include simultaneously forming a hydrogel precursor film and pouch construct by dipping a mold such as a rod into a polymer solution to solution cast the hydrogel precursor film onto the mold as depicted in step 420. In some embodiments, solution casting includes removing the mold, coated in polymer, from the polymer solution, and allowing the polymer coating to dry on the mold. In some embodiments, the dried polymer can be soaked in water to swell the polymer film before detaching the polymer film from the mold. In some embodiments, the polymer can be selected from any biocompatible polymer. In some embodiments, the polymer can be selected from a polyurethane, an acrylate, a polyethyleneglycol, a polydimethylsiloxane, a polyisobutylene, and combinations thereof. In some embodiments, the polymer solution can include about 80 wt.-% to about 95 wt.-% ethanol (e.g., about 85 wt.-% to about 90 wt.-%, about 90 wt.-% to about 95 wt.-%, about 80 wt.-% to about 90 wt.-% ethanol, about 80 wt.-%, about 85 wt.-%, about 90 wt.-%, or about 95 wt.-%) and about 8 wt.-% to about 16 wt.-% (e.g., about 8 wt.-% to about 10 wt.-%, about 10 wt.-% to about 12 wt.-%, about 12.5 wt.-% to about 14 wt.-%, about 14 wt.-% to about 16 wt.-%, about 8 wt.-%, about 10 wt.-%, about 12 wt.-%, about 12.5 wt.-%, about 14 wt.-%, or about 16 wt.-%) of a polyurethane. The precursor film or pouch can then be soaked in a solution including monomer, initiator, and crosslinker to form a soaked hydrogel precursor film as in step 421. The soaked hydrogel precursor film can then be polymerized in step 422 to produce an encapsulation device 424. In some embodiments, the pouch can have more than one opening. Optionally, if required prior to filling, one or more openings can be sealed, as in step 423, to produce the encapsulation device 424. The device formed by this process includes a continuous hydrogel device. The device can optionally be filled with cells or other material in step 425 through an opening or port in the hydrogel construct. Upon filling with cell or other material, the device can be sealed in step 426 to produce a filled, sealed encapsulation device or implant. The device can then optionally be implanted in step 427 into a subject by known methods of implantation.

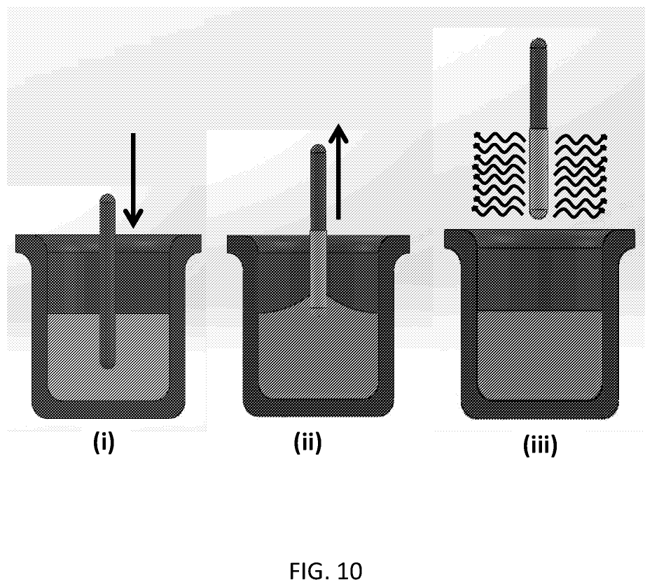

An example of a schematic of forming a device by dipping a mold such as a rod into a polymer solution to solution cast the hydrogel precursor film onto the mold is shown in FIG. 10.

The hydrogel precursor film can be formed, for example, by solution casting a hydrogel precursor film. In some embodiments, solution casting comprises pouring a polymer solution of a defined concentration over a known area of a mold and allowed to dry. After drying, the film is soaked in water to swell and detach from the mold. The polymer solution volume per unit area at a given concentration determines the final film thickness. In some embodiments, solution casting can include immersing at least a portion of a mold in a polymer solution. The mold can then be removed from the solution and the film of solution coating the dipped portion of the mold is allowed to dry. The dried film is then soaked in water and detached from the mold, thereby forming a continuous hydrogel precursor film having one or more openings.

The hydrogel precursor film can be formed, for example, by pouring a polymer solution of a defined concentration into a mold. The polymer solution can be allowed to dry in the mold. After drying, the film is soaked in water to swell and detach from the mold. The polymer solution volume per unit area at a given concentration determines the final film thickness. A hydrogel precursor film, can be formed, for example, into sheets. For example, two sheets of approximately equal size can be made into a pouch by partially sealing (e.g., sealing all but one side, or all but a portion of 1 or 2 sides) with a physical or chemical seal. For example, one sheet can be folded and partially sealed on the non-folded sides (e.g., sealed on all but one side, or all but a portion of 1 or 2 sides) with a physical or chemical seal. In some embodiments, a separator can be used between pieces of a hydrogel precursor film to define the cavity when forming a hydrogel construct. In some embodiments, the separator can be removed after forming the hydrogel construct.

In some embodiments, a hydrogel precursor film can be formed from more than one hydrogel with a single hydrogel network. Without being bound by any particular theory, it is believed that using more than one hydrogel to form a hydrogel precursor (e.g., the same monomer with different average water content) can allow for fine-tuning properties (e.g., water content, thickness, tensile modulus, or ability of solutes to diffuse through the hydrogel) of the hydrogel precursor film that is produced. For example, a mixture of two hydrogels can be used to form a hydrogel precursor film with a single hydrogel network. In some embodiments, the two hydrogels are selected from the group consisting of a polyurethane hydrogel having an average water content of 60%, a polyurethane hydrogel having an average water content of 70%, a polyurethane hydrogel having an average water content of 80%, and a polyurethane hydrogel having an average water content of 90%. In some embodiments, the two hydrogels can be mixed in a 1:1 (i.e., 50:50) ratio. In some embodiments, the two hydrogels can be mixed in a 3:1 (i.e., 75:25) ratio.

The devices include hydrogel films having integral zwitterionic functional groups, such as phosphocholine or betaine (e.g., sulfobetaine or carboxybetaine) groups. Without wishing to be bound by any particular theory, it is believed the zwitterionic groups can provide high surface hydrophilicity and low protein binding, and increase host outcomes by through reduced inflammation, phagocytosis, protein adsorption and thrombus formation when implanted in vivo.

In some embodiments, the polymerized hydrogel film consists essentially of hydrogel. Thus, the encapsulation device, in some embodiments, does not contain any other supporting structures such as hard plastic films or mesh networks to impart mechanical properties on the device. In some embodiments, the polymerized hydrogel film does not contain biological cells. In some embodiments, the hydrogel films, hydrogel constructs, and the encapsulation devices are biocompatible.

In some embodiments, the devices include multiple network hydrogels. Multiple network hydrogels are composite hydrogels with two or more interpenetrating networks. Without wishing to be bound by theory, it is believed multiple network architecture results in a synergistic effect with higher mechanical properties than component individual networks. The films and constructs described herein exhibit high fracture energy and modulus.

In some embodiments, physical blends of two or more hydrogels mixed together can be used to create the hydrogel constructs.

In some embodiments, the hydrogel films and constructs are operable to allow permeation of solutes such as cellular products and nutrients while protecting any material contained within the cavity from immune attack when implanted in vivo. Permeability of the hydrogel film can be controlled by controlling the crosslinking density of the hydrogel film, including the multiple network film, controlling equilibrium water content (EWC) of the hydrogel, and blending multiple hydrogels together. In some embodiments, the hydrogel film is operable to allow permeation of solutes having a size of about 100 kD or less. In some embodiments, the hydrogel film is operable to allow permeation of solutes having a size of from about 1 kD to about 50 kD.

In some embodiments, the thickness of the hydrogel film can be varied to allow variation in the permeability of the construct. In some embodiments, the diffusion rate of cellular products can be controlled by varying the thickness of the hydrogel film. For example, in some embodiments, increasing the thickness of the hydrogel film increases the resistance to diffusion of cellular products contained within the cavity of the construct. In some embodiments, the encapsulation efficiency of the device can be increased by decreasing the thickness of the hydrogel film.

In some embodiments, the hydrogel construct has a thickness of from about 10 μm to about 1.5 mm, about 10 μm to about 1 mm, about 10 μm to about 600 μm, about 10 μm to about 300 μm, from about 20 μm to 210 μm, from about 10 μm to 110 μm, from about 20 μm to 80 μm, from about 50 μm to about 150 μm, from about 90 μm to about 110 μm, or from about 120 μm to about 180 μm. In some embodiments, the hydrogel construct has a thickness of less than 50 μm. In some embodiments, the hydrogel construct has a thickness of from about 10 μm to about 50 μm.

In some embodiments, the hydrogel construct is formed by dip coating. In some embodiments, the hydrogel construct includes a gradient in the film thickness along the length of the construct. In some embodiments, the variation the thickness of the film may vary along its length by 20-30 μm. In some embodiments, the hydrogel film exhibits a uniform thickness across its length. In some embodiments, the hydrogel construct is formed by spray coating or electrospinning.

In some embodiments, the hydrogel films have an average water content of from about 40% to about 99%, as determined by thermogravimetry. In some embodiments, the water content can be varied to adjust the permeability of the construct. In some embodiments, higher levels of water content in the hydrogel films result in higher levels of permeability in the construct as compared to constructs comprising hydrogel films having lower levels of water content.

In some embodiments, the devices comprise a seal or sealing member that seals or closes the one or more openings in the hydrogel construct. In some embodiments, the seal or sealing member is a physical seal such as a clip, a plug, or a knot tied in an external material such as string or thread. In some embodiments, the seal or sealing member is a chemical seal, such as glue, crosslinking, or bonding to one or more additional substrates. In some embodiments, the seal can include a plug, such as a silicone plug, having an integral port to provide easy loading of the cavity without damage to the hydrogel. In some embodiments, the plug can be chemically bonded to the hydrogel film. The plug can be coated with an initiator and brought into contact with a hydrogel precursor film that has been soaked in monomer and crosslinker solution to form a soaked hydrogel precursor film. When the film is polymerized, the initiator-coated plug will polymerize to the film, thus closing the cavity while also providing a loading and/or refilling port in the device.

The macro-sized encapsulation devices disclosed herein can provide high loading capacity for material, such as biological material or biological cells. In some embodiments, the cavity has a volume of from about 1 μl to about 5 ml. In some embodiments, the cavity has a volume of from about 1 μl to about 1 ml. In some embodiments, the cavity has a volume of about 1 μl about 2 μl about 5 μl about 10 μl, about 20 μl, about 100 μl, about 500 μl, about 1 ml, about 2 ml, or about 5 ml.

In some embodiments, the cavity is operable to receive material such as biological material or cells. In some embodiments, the cavity of the device is partially or fully filled with biological material through the one or more openings in the hydrogel construct and the one or more openings can then be sealed. In some embodiments, the biological material positioned within the cavity can be biological cells. In some embodiments, the biological material positioned within the cavity can include a cell suspension. The cell suspension can, in some embodiments, include nutrients, drugs, components that aid cell survival, and/or cell matrix.

In some embodiments, the material positioned within the cavity can comprise a therapeutic such as a protein (e.g., an antibody, an antibody-drug conjugate, or an enzyme) or small molecule (e.g., an anticancer drug). In some embodiments, the material positioned in the cavity can comprise a therapeutic mixed with or conjugated to one or more additional molecules to limit diffusion through the device (e.g., a PEG). In some embodiments, the material positioned in the cavity can be modified such that it is activated or available to diffuse through the hydrogel over a certain period of time (e.g., 1 month, 2 months, 4 months, 6 months, or 1 year). Without being bound by any particular theory, it is believed that local administration of therapeutics allows for lower dosing, and fewer side effects as a result.

In some embodiments, the biological cells positioned within the cavity of the device can produce a therapeutic, such as a protein (e.g., an antibody or an enzyme) or a small molecule (e.g., hormone, a therapeutic natural product). In some embodiments, the hormone can be a peptide hormone. In some embodiments, the peptide hormone is insulin. In some embodiments, the peptide hormone is glucagon. In some embodiments, the biological cells are engineered to produce a therapeutic. In some embodiments, the biological cells can produce a protein that is not produced in a functional form or in an adequate amount in a human or an animal with a disorder. In some embodiments, the disorder is an inherited metabolic disorder. For example, the biological cells can produce proteins associated with a disease selected from: diabetes, including type 1 diabetes, type 2 diabetes, gestational diabetes, other forms of diabetes, and other diseases in which a protein is not produced in a functional form or is not produced in an adequate amount, e.g., adrenal insufficiency and the like. In some embodiments, the protein is engineered to be exported from the biological cells. In some embodiments, the protein can have an effect on target cells without being internalized to the target cells. In some embodiments, the protein is engineered to be imported into target cells.

In some embodiments, the biological cells produce 2 or more (e.g., 3, 4, or 5) therapeutics. In some embodiments, the 2 or more therapeutics are produced by a single type of cell (e.g., a cell that produces or is engineered to produce 2 or more therapeutics). In some embodiments, the 2 or more therapeutics are produced by more than 1 type of cell (e.g., two types of cells, each producing or engineered to produce 1 therapeutic).

In some embodiments, the biological cells positioned within the cavity of the device can be pancreatic islet cells. Type 1 diabetes (T1D) is an immune mediated disease characterized by destruction of islet cells and affecting millions of people worldwide. With advances in field of stem cells, large scale well characterized allogenic beta islet cells may be produced. Encapsulation of allogenic cells is one method of protecting the donor cells from immune attack once inside the host. Encapsulation with the devices described herein may avoid the need for immune suppression in host subjects. Islet cell implantation can also be therapeutic in individuals with type 2 diabetes. Implantation of islet cells in the devices described herein can provide therapeutic levels of insulin produced by cells contained within the cavity of the device and permeating through the hydrogel film to individuals with type 1 or type 2 diabetes.

The document also provides a method for implanting cells into a patient in need thereof comprising implanting into the patient a cell-containing encapsulation device as described herein.

In some embodiments, the cell-containing encapsulation device can contain cells of at least one cell type selected from induced pluripotent stem cells (IPSC), insulin-secreting beta islet cells, glucagon-secreting alpha islet cells, and combinations thereof.

In some embodiments, the cell-containing encapsulation device can further contain matrix and/or nutrients to support the cells contained within the device.

In some embodiments, the devices are expandable such that over-filling the cavity causes the device to expand. In some embodiments, the devices are refillable.

In some embodiments, the devices are fabricated so as to be visible through medical imaging techniques (e.g., x-ray, PET, CT, or MRI). For example, a device can be impregnated with, filled with, or attached to a radiopaque substance or tag.

The document further provides methods for treating a disease in a patient comprising implanting into a patient in need thereof a cell-containing encapsulation device as described herein. In some embodiments, the disease can be type 1 diabetes. In some embodiments, the disease can be type 2 diabetes. In some embodiments, the disease can be other forms of diabetes. In some embodiments, the disease can be a disease of the adrenal gland.

The document further provides methods for preventing immunologic response to implanted biological material such as cells. The methods can comprise encapsulating the biological material into the devices described herein and implanting the device into a subject in need thereof.

A number of embodiments of the invention have been described. Nevertheless, it will be understood that various modifications can be made without departing from the spirit and scope of the invention. For example, the composition of the hydrogels can be varied to produce appropriate permeation and strength properties for the hydrogel film depending on the desired implantation location and treatment type. Accordingly, other embodiments are within the scope of the following claims.

### EXAMPLE EMBODIMENTS

Embodiment 1 is a device comprising:

a hydrogel construct including a hydrogel film having integral zwitterionic functional groups;

a cavity defined by said construct; and

one or more openings in said construct.

Embodiment 2 is the device of embodiment 1, wherein said hydrogel film comprises a multiple network hydrogel.

Embodiment 3 is the device of embodiment 2, wherein said hydrogel film comprises a double network hydrogel.

Embodiment 4 is the device of any one of embodiments 1-3, wherein said hydrogel film does not contain biological cells.

Embodiment 5 is the device of any one of embodiments 1-4, wherein said hydrogel film consists essentially of hydrogel.

Embodiment 6 is the device of any one of embodiments 1-5, wherein said hydrogel construct is operable to allow permeation of solutes having a size of 100 kD or less through said hydrogel film while minimizing permeation of solutes having a size greater than 100 kD.

Embodiment 7 is the device of any one of embodiments 1-6, wherein said hydrogel construct has a thickness of from about 10 μm to about 300 μm.

Embodiment 8 is the device of any one of embodiments 1-6, wherein said hydrogel construct has a thickness of from about 10 μm to about 110 μm.

Embodiment 9 is the device of any one of embodiments 1-6, wherein said hydrogel construct has a thickness of from about 20 μm to about 80 μm.

Embodiment 10 is the device of any one of embodiments 1-9, wherein said cavity has a volume of from about 1 μl to about 5 ml.

Embodiment 11 is the device of any one of embodiments 1-10, wherein said hydrogel film comprises one or more polymers selected from a polyurethane, an acrylate, a polyethyleneglycol, a polydimethylsiloxane, a polyisobutylene, a hydrogel having phosphocholine groups, a hydrogel having betaine groups, and combinations thereof.

Embodiment 12 is the device of any one of embodiments 1-11, further comprising one or more sealing members operable to close at least one of said openings.

Embodiment 13 is the device of embodiment 12, wherein said one or more sealing members are selected from a physical seal and a chemical seal.

Embodiment 14 is the device of embodiment 13, wherein said one or more sealing members is a physical seal comprising a clip, a plug, or a knot.

Embodiment 15 is the device of embodiment 13, wherein said one or more sealing members is a chemical seal selected from a glue and a chemical bond.

Embodiment 16 is the device of any one of embodiments 1-15, wherein said hydrogel film comprises two or more hydrogel layers chemically linked through at least one contact portion.

Embodiment 17 is the device of any one of embodiments 1-15, wherein said hydrogel film comprises a continuous hydrogel layer.

Embodiment 18 is the device of any one of embodiments 1-17, further comprising biological material positioned within said cavity.

Embodiment 19 is the device of embodiment 18, wherein said biological material comprises biological cells.

Embodiment 20 is the device of embodiment 18, wherein said biological material comprises cells of at least one cell type selected from induced pluripotent stem cells (IPSC), insulin-secreting beta islet cells, glucagon-secreting alpha islet cells, and combinations thereof.

Embodiment 21 is the device of embodiment 18, wherein said biological material comprises cells that produce a therapeutic protein.

Embodiment 22 is the device of embodiment 18, wherein said biological material comprises cells that produce a protein associated with a disease selected from: diabetes, including type 1 diabetes, type 2 diabetes, gestational diabetes, other forms of diabetes, and adrenal insufficiency.

Embodiment 23 is the device of embodiment 18, wherein said biological material comprises cells that produce a hormone.

Embodiment 24 is the device of embodiment 23, wherein the hormone is a peptide hormone.

Embodiment 25 is the device of embodiment 18, wherein said biological material comprises cells that produce insulin, glucagon, or a combination thereof.

Embodiment 26 is a process for forming an encapsulation device comprising:

forming a hydrogel precursor film into a pouch having a cavity defined by said hydrogel precursor film and one or more openings in said hydrogel precursor film in fluid communication with said cavity.

Embodiment 27 is the process of embodiment 26, wherein said encapsulation device comprises integral zwitterionic functional groups.

Embodiment 28 is the process of any one of embodiments 26-27, wherein said encapsulation device comprises a multiple network hydrogel film.

Embodiment 29 is the process of any one of embodiments 26-28, wherein forming said hydrogel precursor film comprises casting a polymer selected from a polyurethane, an acrylate, a polyethyleneglycol, a polydimethylsiloxane, and a polyisobutylene.

Embodiment 30 is the process of any one of embodiments 26-29, further comprising:

soaking the hydrogel precursor film in a solution comprising a monomer, a crosslinker, and an initiator to form a soaked hydrogel precursor film; and

polymerizing the soaked hydrogel precursor film to form a hydrogel film.

Embodiment 31 is the process of any one of embodiments 26-30, wherein said encapsulation device is biocompatible.

Embodiment 32 is the process of any one of embodiments 26-31, wherein said cavity is operable to receive biological material.

Embodiment 33 is the process of any one of embodiments 26-32, further comprising filling said cavity with biological material.

Embodiment 34 is the process of embodiment 33, wherein said biological material comprises biological cells.

Embodiment 35 is the process of embodiment 33, wherein said biological material comprises cells of at least one cell type selected from induced pluripotent stem cells (IPSC), insulin-secreting beta islet cells, glucagon-secreting alpha islet cells, and combinations thereof.

Embodiment 36 is the process of embodiment 33, wherein said biological material comprises cells that produce a therapeutic protein.

Embodiment 37 is the process of embodiment 33, wherein said biological material comprises cells that produce a protein associated with a disease selected from: diabetes, including type 1 diabetes, type 2 diabetes, gestational diabetes, other forms of diabetes, and adrenal insufficiency.

Embodiment 38 is the device of embodiment 33, wherein said biological material comprises cells that produce a hormone.

Embodiment 39 is the device of embodiment 38, wherein the hormone is a peptide hormone.

Embodiment 40 is the device of embodiment 33, wherein said biological material comprises cells that produce insulin, glucagon, or a combination thereof.

Embodiment 41 is the process of any one of embodiments 26-40, further comprising sealing said one or more openings.

Embodiment 42 is the process of embodiment 41, wherein said sealing comprises physically or chemically sealing said one or more openings.

Embodiment 43 is the process of any one of embodiments 26-42, wherein forming a hydrogel film comprises solution casting said hydrogel film.

Embodiment 44 is the process of any one of embodiments 26-43, wherein forming said hydrogel film into a pouch comprises solution casting said hydrogel on a mold by immersing at least a portion of said mold in a hydrogel solution to, thereby forming a continuous hydrogel film having one or more openings.

Embodiment 45 is the process of any one of embodiments 26-44, wherein forming said hydrogel film into a pouch comprises sandwiching a separator between a first surface of a first hydrogel film and a first surface of a second hydrogel film such that a first portion of said first surface of said first hydrogel film does not contact a first portion of said first surface of said second hydrogel film and a second portion of said first surface of said first hydrogel film contacts a second portion of said first surface of said second hydrogel film.

Embodiment 46 is the process of embodiment 45, further comprising removing said separator.

Embodiment 47 is the process of any one of embodiments 26-46, wherein said hydrogel film has a thickness of from about 10 μm to about 300 μm.

Embodiment 48 is the process of any one of embodiments 26-46, wherein said hydrogel film has a thickness of from about 10 μm to about 110 μm.

Embodiment 49 is the process of any one of embodiments 26-46, wherein said hydrogel film has a thickness of from about 90 μm to about 110 μm.

Embodiment 50 is the process of any one of embodiments 26-46, wherein said hydrogel film has a thickness of from about 20 μm to about 80 μm.

Embodiment 51 is the process of any one of embodiments 26-50, wherein said cavity has a volume of from about 1 μl to about 5 ml.

Embodiment 52 is the process of any one of embodiments 26-51, further comprising inserting biological cells into said cavity.

Embodiment 53 is the process embodiment 52, further comprising sealing said cavity with a chemical or physical seal.

Embodiment 54 is a method for implanting cells into a patient comprising: implanting into a patient in need thereof the device of any one of embodiments 18-24.

Embodiment 55 is a method for treating a disease comprising implanting into a patient in need thereof the device of any one of embodiments 18-24.

Embodiment 56 is the method of embodiment 55, wherein said disease is type 1 diabetes.

Embodiment 57 is the method of embodiment 55, wherein said disease is type 2 diabetes.

Embodiment 58 is a method of preventing immunologic attack on implanted biological material comprising encapsulating said biological material into the device of any one of embodiments 1-17 and implanting said device into a subject in need thereof.

Embodiment 59 is the device of any one of embodiments 18-24, wherein the device is expandable.

Embodiment 60 is the device of any one of embodiments 18-24, wherein the device is refillable.

### EXAMPLES

Hydrogel membranes and encapsulation devices were fabricated with the hydrogel compositions set forth below, by solution casting on a ¼ inch rod dipped in 12.5 wt.-% polymer.

| Hydrogel | Composition | Blend Ratio |
|---|---|---|
| HG60 | Polyurethane hydrogel having an | n/a |
| | average water content of 60% | |
| HG70 | Polyurethane hydrogel having an | n/a |
| | average water content of 70% | |
| HG80 | Polyurethane hydrogel having an | n/a |
| | average water content of 80% | |
| HG90 | Polyurethane hydrogel having an | n/a |
| | average water content of 90% | |
| BG90/80(Also called | Blend of HG90 and HG80 | 1:1 |
| HG90/80 or | | |
| HG90/HG80) | | |
| (50:50 or 1:1) | | |
| BG80/70 (Also called | Blend of HG80 and HG70 | 3:1 |
| HG80/70 or | | |
| HG80/HG70) | | |
| (75:25 or 3:1) | | |
| BG80/70 | Blend of BG80 and BG70 | 1:1 |
| (50:50 or 1:1) | | |

### Example 1: Hydrogel Membrane Properties

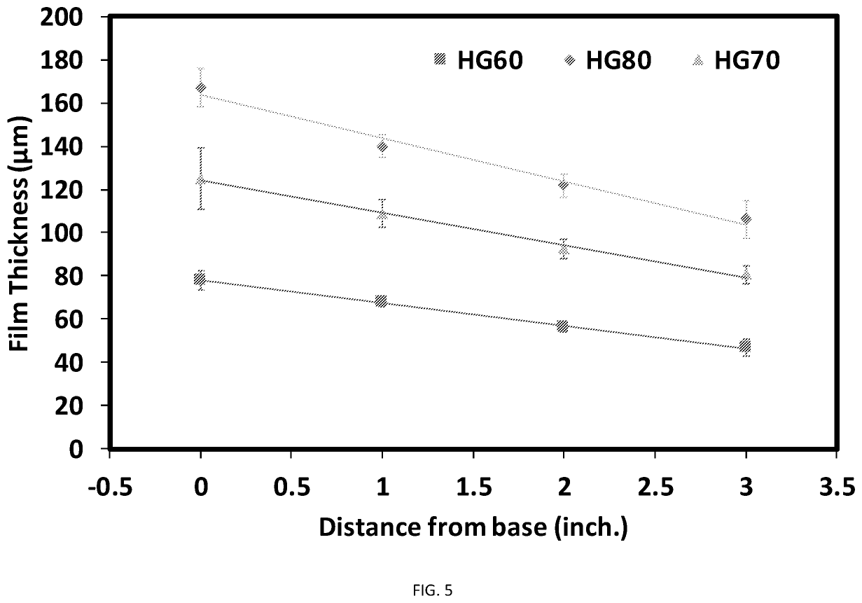

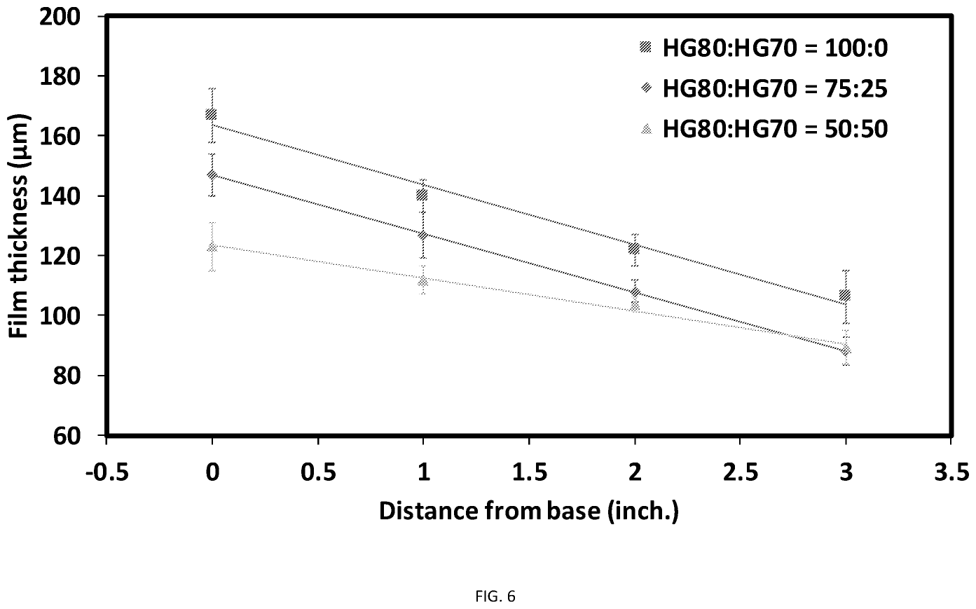

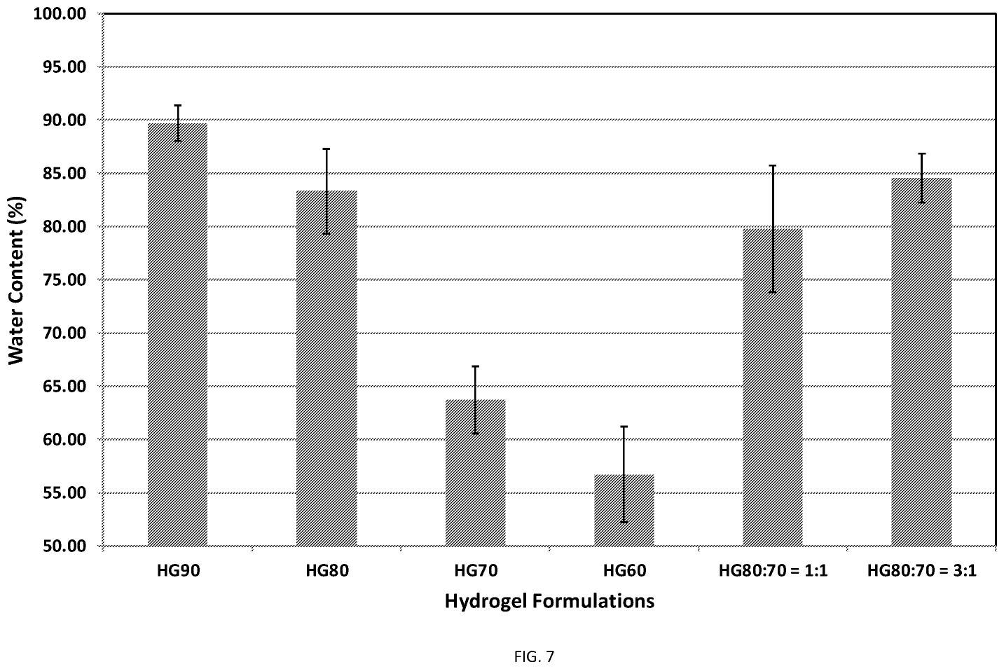

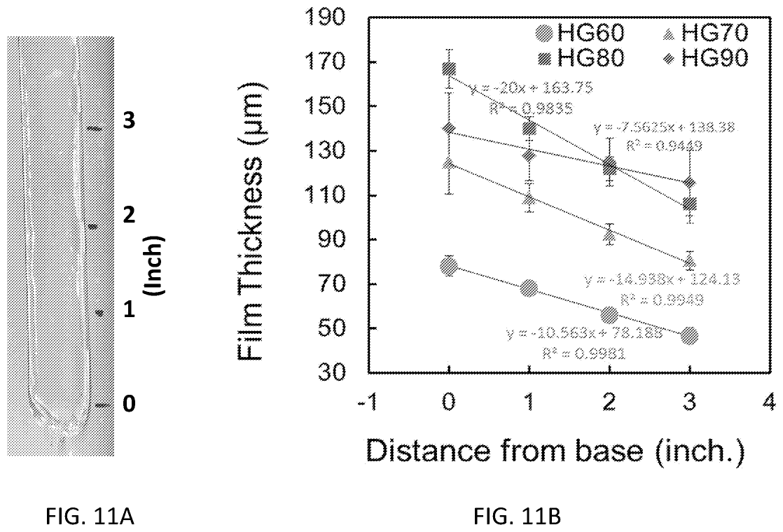

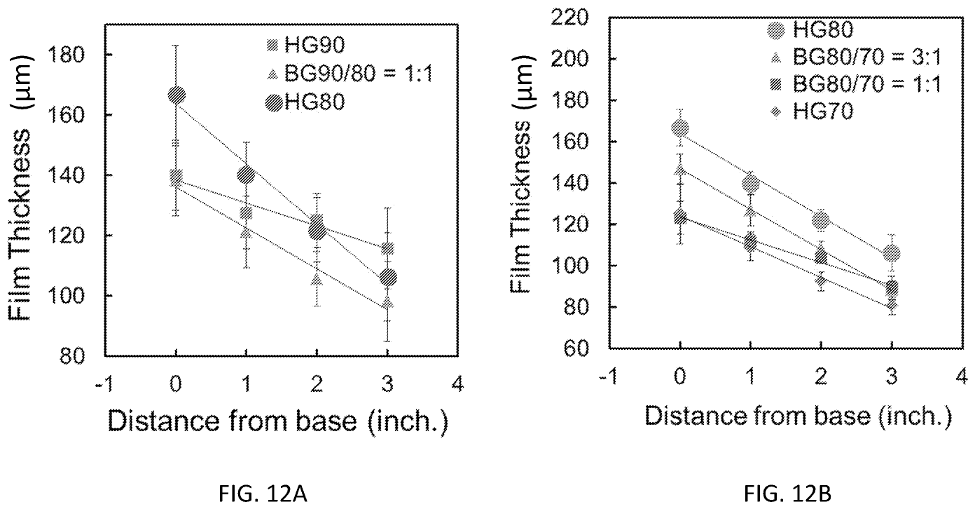

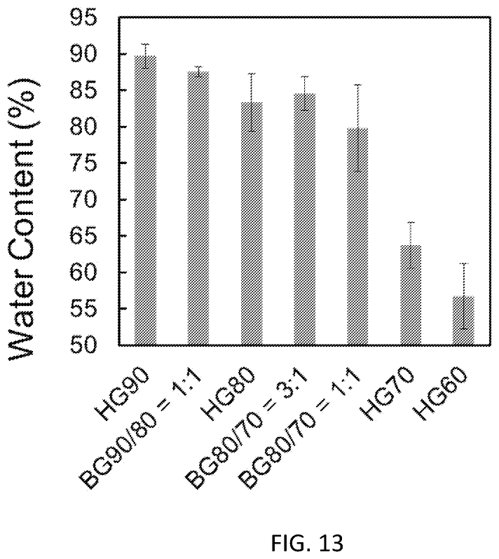

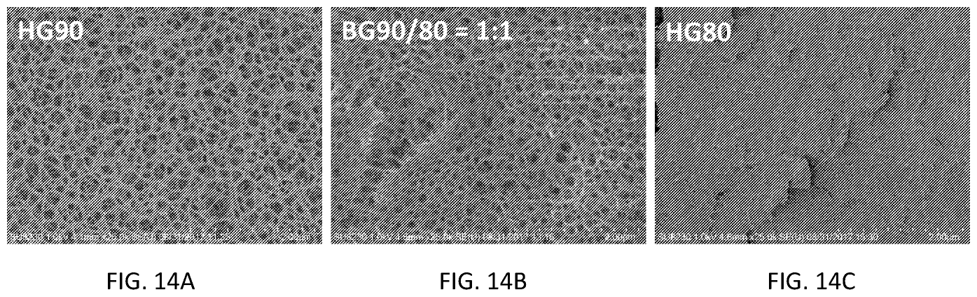

The membranes were fabricated and membrane thickness and water content was evaluated. The membrane thickness of each fabricated membrane is shown in FIGS. 5 and 6. The membrane thickness of additional membranes is shown overlaid with the data from FIG. 5 in FIG. 11B, which includes HG90. An example of a hydrogel fabricated by dip coating is shown in FIG. 11A. The membrane thickness of blended membranes including those shown in FIG. 6 are shown with their respective components in FIG. 12A (including BG90/80) and FIG. 12B (including BG80/70). The water content of each fabricated membrane is shown in FIG. 7. The water content of additional membranes is shown overlaid with the data from FIG. 7 in FIG. 13 (including BG90/80=1:1). Cryo-SEM images of exemplary membranes are shown in FIGS. 14A, 14B, and 14C.

### Example 2: Dye Diffusion Study

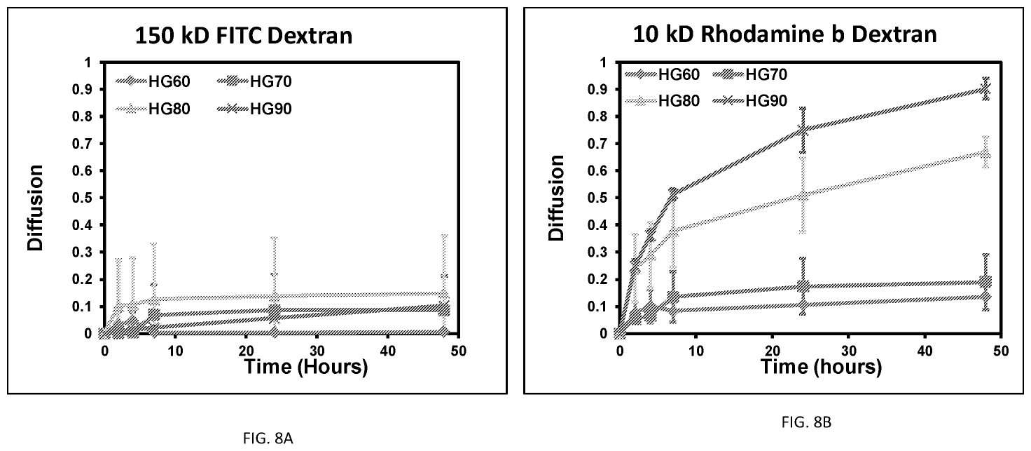

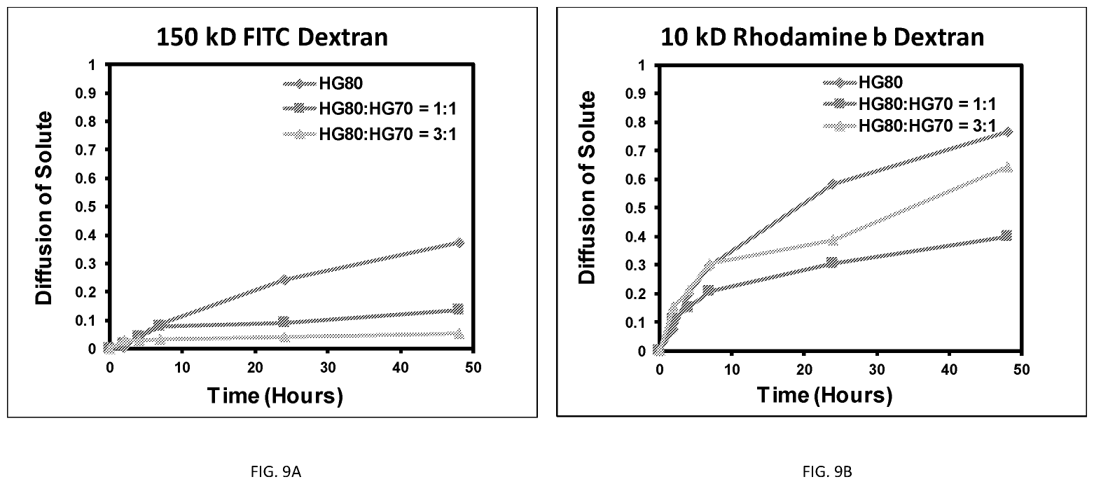

The membranes were formed into encapsulation devices and loaded with a mixture of approximately 1 mg/ml FITC-Dextran (MW 150 kD) and approximately 1 mg/ml Rhodamine B-Dextran (MW 10 kD) in 1× phosphate buffered saline (PBS). 1 ml of the dye solution was added to each device and soaked in 15 mL PBS at 37° C. on an orbital shaker for 48 hours. Aliquots of the soaking solution were removed at 2, 4, 7, 24, and 48 hours and analyzed. The results are shown for non-blended membranes in FIG. 8A, 8B, and for blended membranes in FIGS. 9A, and 9B.

### Example 3: Effect of Catalyst Removal on Cell Viability

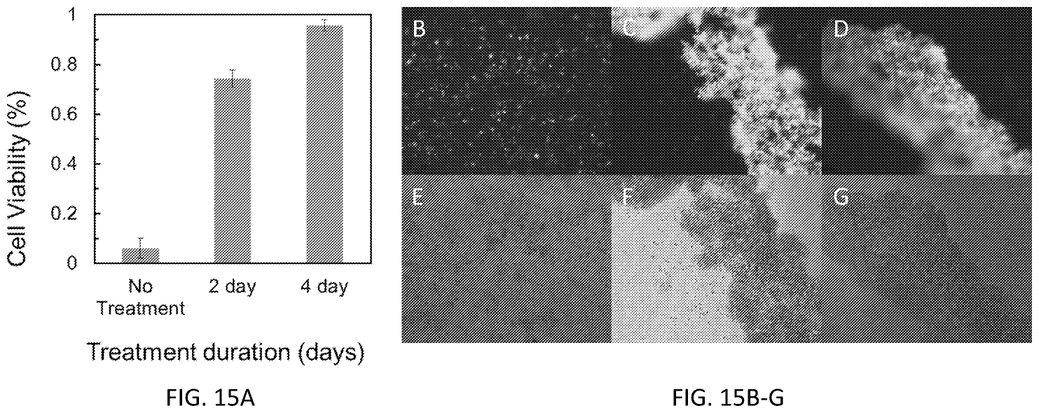

Catalyst is used for synthesis of polyurethanes which can be toxic to the encapsulated cells. Fabricated membranes were treated with 10% ethanol solution to remove the catalyst. Membranes were soaked for a given time period and sampled to evaluate the effect of treatment duration on cell viability. Green fluorescent protein expressing MIN-6 cells were encapsulated in the membranes and the viability was measured after two days of incubation. The results, as percent viability, are shown in FIG. 15A. The images from microscopy are shown as (fluorescent, bright field pairs) in FIG. 15B-G, with FIGS. 15B and 15E corresponding to treatment of 0 days, FIGS. 15C and 15F corresponding to treatment of 2 days, and FIGS. 15D and 15G corresponding to treatment of 4 days.

### Example 4: Effect of Zwitterionic Content and Type of Zwitterion Monomer on Hydrogel Water Content

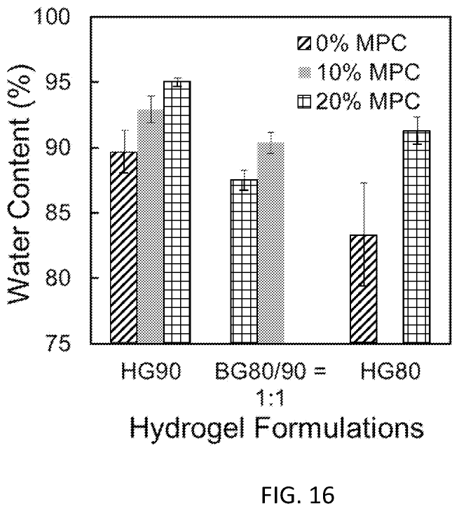

The membranes were fabricated into double networks by polymerizing 0%, 10%, and 20% of zwitterionic monomer MPC to form the second network. Incorporation of second monomer and formation of double network is confirmed by change in the water content of the hydrogels. The water content of each fabricated double network is shown in FIG. 16.

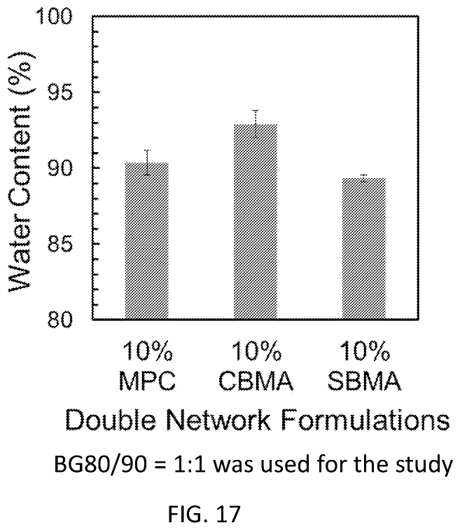

The double network membranes were also fabricated using 10% each of MPC, CBMA, and SBMA monomer to form the second network with BG90/80=1:1 as the first network. The water content of each fabricated double network is shown in FIG. 17.

### Example 5: Mechanical Properties of Hydrogel and Effect of Sterilization

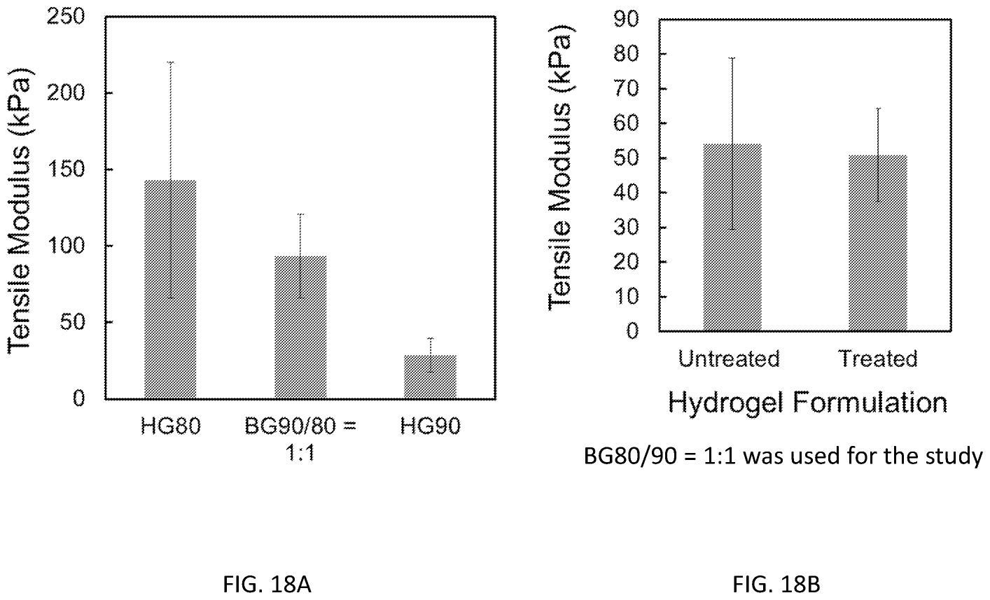

To evaluate the effect of blending on the modulus of the hydrogel, membranes were fabricated and samples were cutout using a punch. Modulus was evaluated in tensile mode and the results are shown in FIG. 18A.

To evaluate the effect of sterilization on the membrane, modulus was evaluated for both treated (supercritical carbon dioxide, Novasterilis) and untreated hydrogel membranes of the formulation BG80/90=1:1. The tensile modulus of each fabricated membrane is shown in FIG. 18B.

### Example 6: Protein Adsorption

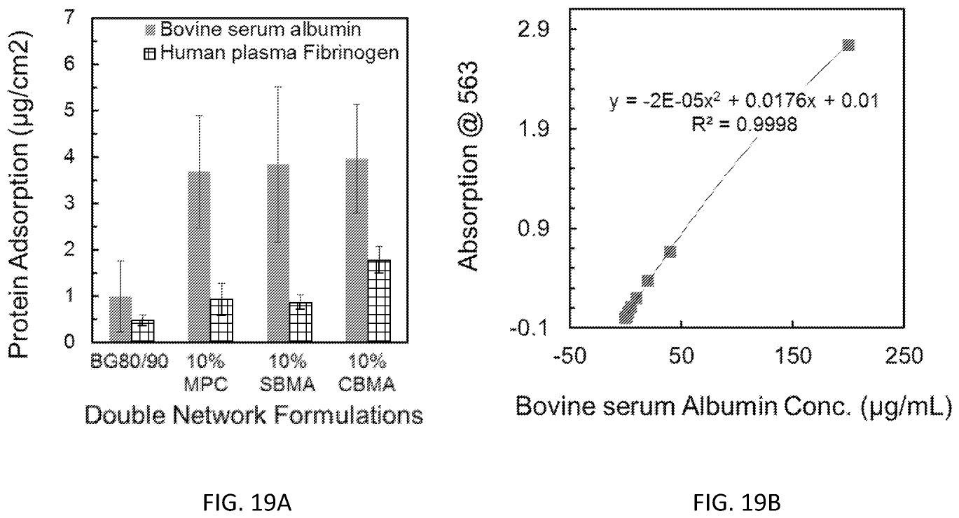

The membranes were fabricated and incubated with protein solutions for 1 hour at 37° C. After the incubation, the samples were washed with PBS and incubated in 1.5 SDS solution for 2 hours at 37° C. SDS solution was analyzed by MicroBCA assay to determine the protein adsorbed using BSA as standard. Results are shown in FIG. 19B.

Both bovine serum albumin (BSA) and human plasma fibrinogen were evaluated. The protein adsorption for each fabricated membrane is shown in FIG. 19A.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A device comprising:
a hydrogel construct including a hydrogel film having integral zwitterionic functional groups;
a cavity defined by said construct; and
one or more openings in said construct.

2. The device of claim 1, wherein said hydrogel film comprises a multiple network hydrogel.

3. The device of claim 1, wherein said hydrogel film consists essentially of hydrogel.

4. The device of claim 1, wherein said hydrogel construct has a thickness of from about 10 μm to about 300 μm.

5. The device of claim 1, wherein said cavity has a volume of from about 1 μl to about 5 ml.

6. The device of claim 1, wherein said hydrogel film comprises one or more polymers selected from a polyurethane, an acrylate, a polyethyleneglycol, a polydimethylsiloxane, a polyisobutylene, a hydrogel having phosphocholine groups, a hydrogel having betaine groups, and combinations thereof.

7. The device of claim 1, further comprising biological material positioned within said cavity.

8. The device of claim 7, wherein said biological material comprises cells of at least one cell type selected from induced pluripotent stem cells (IPSC), insulin-secreting beta islet cells, glucagon-secreting alpha islet cells, and combinations thereof.

9. A process for forming an encapsulation device comprising:
forming a hydrogel precursor film into a pouch having a cavity defined by said hydrogel precursor film and one or more openings in said hydrogel precursor film in fluid communication with said cavity.

10. The process of claim 9, wherein said encapsulation device comprises integral zwitterionic functional groups.

11. The process of claim 9, further comprising:
soaking the hydrogel precursor film in a solution comprising a monomer, a crosslinker, and an initiator to form a soaked hydrogel precursor film; and
polymerizing the soaked hydrogel precursor film to form a hydrogel film.

12. The process of claim 9, wherein said encapsulation device is biocompatible.

13. The process of claim 9, wherein said cavity is operable to receive biological material.

14. The process of claim 9, further comprising filling said cavity with biological material.

15. The process of claim 9, wherein said hydrogel film has a thickness of from about 10 μm to about 300 μm.

16. (canceled)

17. A method for treating a disease comprising implanting into a patient in need thereof the device of claim 7.

18. The method of claim 17, wherein said disease is type 1 diabetes.

19. The method of claim 17, wherein said disease is type 2 diabetes.

20. (canceled)
